(19) United States
(12) Patent Application Publication
(10) Pub. No.: US 2009/0226529 A1
(43) Pub. Date: Sep. 10, 2009

(54) GRANULATED COMPOSITION

(75) Inventors: Christian Quellet, Biel (CH); Johnny Bouwmeesters, Oetwil am see (CH); Sibilla Delorenzi, Zurich (CH)

Correspondence Address: NORRIS, MCLAUGHLIN & MARCUS, 875 THIRD AVE, 18TH FLOOR, NEW YORK, NY, 10022, US

(73) Assignee: Givaudan SA, Vemier (CH)

(21) Appl. No.: 11/720,010

(22) PCT Filed: Nov. 24, 2005
(86) PCT No.: PCT/CH2005/000696

§ 371 Date: Aug. 1, 2007

(30) Foreign Application Priority Data
Nov. 24, 2004 (GB) 0425795.2

(51) Int. Cl. A61K 9/50; C11D 3/50; C11D 17/06

(52) U.S. Cl. 424/493; 424/497; 510/349

(57) ABSTRACT

A method of making a granulated composition that comprises frangible microcapsules containing at least one active ingredient, such as a fragrance, comprising the application of discrete droplets of an aqueous slurry of the microcapsules on to a non-fluidised bed of powdered material, and then drying, the powdered material having a surface tension as defined by an initial contact angle of slurry with powder of at least 40°, which contact angle does not change by more than 10% within the first 3 seconds of application of slurry to powder material.

The granulates made by this method are unusually resilient and can withstand rough treatment (for example, during manufacture of products in which they are incorporated), yet can readily release their contents when required. They are also able to be electrically charged.

This invention relates to granulated compositions comprising water-dispersible microcapsules that contain microencapsulated active ingredients, and to a process of preparing such compositions.

By “active ingredient” is meant any ingredient whose initial encapsulation and later release is desired. It is well known that active ingredients such as fragrances, flavours, insecticides, malodour-counteracting substances, fungicides and mildewcides may be encapsulated in microcapsules comprising a solid shell or membrane, which protects them from their immediate environment and acts as means for their controlled release. A popular and convenient method of producing such encapsulated formulations consists of dispersing the ingredient in a liquid and creating a polymeric membrane on the surface of the droplets. Examples of suitable processes include the coacervation of gelatine with gum Arabic followed by cross-linking with glutaraldehyde. More generally, many polymers or mixtures of polymers capable of forming insoluble complexes under specific conditions can be used to form such interfacial membranes by so-called polymer phase separation processes.

Alternatively, interfacial membranes can be produced by the interfacial polycondensation of various co-monomers and macromers. The polycondensation of melamine with formaldehyde to form so-called aminoplast microcapsules is the most popular of these processes. However, microcapsules having polyester, polyamide or polyurethane membranes are also well known.

These established processes essentially convert emulsions consisting of a dispersed oil phase containing the active ingredient to be encapsulated and a continuous water phase into a suspension of solid microcapsules consisting of a core surrounded by a membrane. Similarly, dispersions and suspensions of solid ingredients in water can be coated with such membranes. The ideal microcapsule membrane should combine low permeability with respect to the encapsulated material and ease of breaking under the appropriate circumstances. The factors determining the permeability of the membrane are the degree of cross-linking, the extent of polycondensation and the thickness of the membrane. Compositions formed in this manner produce excellent results in terms of high loading of ingredients and efficient release when mechanically broken by the effect of excessive shear stresses.

However, the art has paid very little attention to the essential step of incorporating such microcapsules in detergent powders and tablets. Similarly, the question of the stability of these microcapsules during storage in aggressive detergent bases at high relative humidity and high temperature has been widely ignored.

Incorporating any kind of microcapsules into granulated detergent often leads to problems. In particular, the addition of microcapsules of the abovementioned types in the form of aqueous slurries, i.e. in the form in which these microcapsules are obtained after coacervation or interfacial polymerization has been achieved, has the effect of adding appreciable amounts of water to the detergent base, which results in clogging and inhomogeneous distribution or segregation of the microcapsules within the mixture. Furthermore, increasing the humidity around the microcapsules generally accelerates chemical degradation of the microcapsule membrane by the action of free alkaline materials and enzymes.

Converting the microcapsule slurries into powders by conventional drying techniques, such as spray drying, generally results in the production of fine, free-flowing powders containing high levels of potentially highly explosive dust, which may constitute a safety hazard. Furthermore, free-flowing powders having mean particle sizes of less than 100 micrometers are generally not suitable for incorporation into granulated detergents, because of size segregation. Such free-flowing powders have the tendency to accumulate at the bottom of packaging.

Moreover, when spray-dried microcapsules are subjected to high moisture levels and elevated temperature in alkaline media such as detergent powders, they exhibit tendencies to chemical degradation and plasticization. Capsules utilising aldehyde-based cross-linking agents have the additional disadvantage of high levels of free aldehydes being generated during storage, leading to unacceptable levels of, e.g., formaldehyde and glutaraldehyde in the product. This phenomenon is especially observed in conventional spray-dried aminoplast microcapsules. Plasticization leads to leakage of encapsulated ingredients over time and, consequently, to loss of microcapsule performance.

Microencapsulated compositions with desirable traits such as high loading of active ingredients, good chemical stability, low tendency to plasticization when submitted to moisture and alkalinity, low tendency to size segregation when added to products in powder form, and easy handling, can be provided by granulated forms in which the microcapsules are intimately agglomerated. These may typically be produced by conventional agglomeration techniques, such as fluidized bed agglomeration, vortex agglomeration, or combined fluid bed and vortex agglomeration. An alternative way to obtain agglomerates containing microcapsules involves the steps of (i) drying a slurry of microcapsules by spray drying and (ii) suspending the dry microcapsule powder obtained in a fluidized bed and (iii) spraying an aqueous binder under controlled temperature and humidity conditions.

However, when submitted to the conditions of fluidized bed agglomeration by any of the methods described above, microcapsules characterized by a high load of encapsulated active ingredients, and especially of fragrances, and a thin and brittle membrane, cannot usually withstand the mechanical stresses induced by the repeated contacts with neighbouring agglomerates. In particular, the membrane of the microcapsules can be partially or completely destroyed, which leads to significant losses of encapsulated ingredients during the agglomeration process.

It has now been found that it is possible to obtain granulated compositions containing frangible water-dispersible microcapsules having high loads of encapsulated active ingredients, and thin and brittle walls, without affecting the structural integrity of said microcapsules. The granulated composition is free flowing and essentially free of fine particles, and has a pronounced capability of admixing homogeneously with current granulated detergent bases, using standard mixing means such as rotary blenders and vortex mixing units. The composition is chemically stable and has the capability of releasing microcapsules when dispersed in water and wash liquors.

The invention therefore provides a method of making a granulated composition that comprises frangible microcapsules containing at least one active ingredient, comprising the application of discrete droplets of an aqueous slurry of the microcapsules on to a non-fluidised bed of powdered material, and then drying, the powdered material having a surface tension as defined by an initial contact angle of slurry with powder of at least 40°, which contact angle does not change by more than 10% within the first 3 seconds of application of slurry to powder material.

The invention further provides a granulated composition of frangible microcapsules obtainable by a method as hereinabove described.

It has surprisingly been observed that the shape, morphology and size of the agglomerates provided by the process according to the invention depend strongly on the nature of the powder bed. The size depends also on the size of the droplet of slurry applied to the powder bed. Hence, by selecting both drying material and nozzle dimension, it is possible to obtain by simple experimentation a wide range of different types of agglomerates, including sub-millimeter-sized and millimeter-sized spherical agglomerates having a regular or irregular (e.g. orange skin-like) surface, strawberry-like agglomerates, pellets and flakes, as well as biphasic agglomerates characterized by a microcapsule-rich region and a powder-rich region. Some of these are shown in the accompanying figures. Where the microcapsules are dyed, the latter biphasic agglomerates gain aesthetic features of particular interest if a visual cue is desired.

The slurry or dispersion of microcapsules can be applied to the drying bed by any convenient means. It is preferably carried out by spraying, using, for example, pipettes, one-fluid or two-fluid nozzles, mechanically or ultrasonic vibrating nozzles.

Typical examples of materials suitable for use in the powder bed include, but are not limited to
polysaccharides and modified polysaccharides such as starch (e.g. Starch 365, Starch 1415, Starch Mira-Cap®, ex Staley Manufacturing Company) modified starch (e.g. Capsul® or Hi-Cap® 100, ex National Starch), maltodextrin (e.g. IT-6, ex Roquette Frères), modified sugar (e.g. Isomalt F, ex. Palatinit), cellulose, modified cellulose, and mixture thereof,
synthetic polymers such as polyamide powder (e.g. Schaetti Fix-Pulver 140/100-400 my, ex Schaetti AG) and silicone resins (e.g. Tospearl, ex General Electric)
inorganic materials such as porous silica (e.g. Silica gel 60, ex Merck)

The agglomeration process according to the invention does not rely on fluidized bed technologies and therefore does not suffer from the drawbacks previously mentioned. It is based on the surprising discovery that droplets of aqueous slurries containing microcapsules pre-agglomerate spontaneously in contact with specific materials in powder form. By “pre-agglomerate” is meant the formation of a cohesive assembly of small elements having some degree of deformability, owing to the presence of residual water. Pre-agglomerates transform into agglomerates by elimination of this residual water in an oven having temperature ranging from 50 to 200° C.

A particular example of an active ingredient for the purposes of this invention is fragrance, especially fragrance for use in a laundry detergent. The problem in the art is to retain fragrance for release into a wash cycle and not lose a substantial proportion thereof during manufacture and storage, as is often the case. Any fragrance material or combination thereof may be used in this invention. Other examples of active ingredients include biocides, essential oils, cosmetic ingredients, emollients, fabric care ingredients or drugs. It is possible and permissible to have two or more active ingredients present in a composition prepared according to the invention, and the use of the singular “active ingredient” encompasses this possibility.

A preferred feature of this invention is that the granulated composition does not only contain microcapsules containing active ingredients, but that there is also present at least one co-ingredient. By “co-ingredient” is meant a substance that is added to perform some composition-improving function other than that of the active ingredient. Typical particularly useful functions performed by such ingredients include improvement of the dispersion/dissolution of the composition, controlling the release of the microcapsules in wash and rinse liquor, and improving the deposition and anchoring of microcapsules on substrates. Co-ingredients may be chosen from a wide variety of substances including:
Water-soluble filler materials such as inorganic salts, polycarboxylic acids, carbohydrates, and modified carbohydrates;
Salts of mono-, di-, tri- and polycarboxylic acids, especially sodium acetate, citrate and tartrate;
surfactants such as anionic surfactants, nonionic surfactants, cationic surfactants and amphoteric surfactants. Typical surfactants include soaps, alkylbenzene sulfonates, alkyl sulfates and sulfonates, alpha-olefine sulfonates, alpha-sulfo fatty acid esters, alkylether sulfonates, alcohol ethoxylates, alkylphenol ethoxylates, fatty acid alkanolamides, alkylamineoxides, alkyl polyolosides, dialkylammonium chlorides, imidazolinium salts, alkyldimethylbenzylammonium chlorides, esterquats, alkylbetaines, alkylsulfobetaines and surface active polymers such as block and graft copolymers;
disintegrating agents such as water-swellable hydrocolloids. Typical disintegrating agents include modified polycarbohydrates, polyvinylsulfonates and alkali-soluble polymers, e.g. poly((meth)acrylic acid);
poly(maleic acid), poly(alkyl(meth)acrylate-co-(meth)acrylic acid) copolymer, poly(acrylic acid-co-maleic acid)copolymer, poly(methacrylic acid), as well as other acrylic homo- and copolymers thereof;
Deposition promoters such as cationic celluloses, guar gums or chitosan, or polymer containing quaternized amine or imidazoline groups, polyethyleneimines, proteins.

Typical surface active polymers include hydrocolloids such as polysaccharides, modified polysaccharides, gums, proteins, gelatine, polyacrylates, polyacrylamide, polyvinyl sulfonates, polyvinyl alcohols and modified polyvinyl alcohols, especially aceto-acetylated polyvinyl alcohols, PVP (polyvinyl pyrrolidone) Aceto-acetylated polyvinyl alcohols are especially preferred when aminoplast microcapsules are used—they have the very useful function of retarding the undesirable generation of formaldehyde. This will be further discussed hereinunder.

The weight percentage of co-ingredient in the microcapsules may vary from 1% to 80%. The agglomerates of microcapsules, which are the product of the process of this invention, are unique, in that they are sufficiently robust to withstand handling and retain the ingredient, yet are able to release that ingredient efficiently and quickly when desired. It has furthermore surprisingly been found that, under some conditions, the agglomerates produced by the abovementioned process are dispersible in aqueous media, i.e. they have the capability of releasing the individual microcapsules initially added into these media almost spontaneously or with the assistance of a mild mechanical action, such as that taking place in a washing process.

The invention additionally provides a process of releasing into an environment in a controlled manner an ingredient whose presence in that environment is desired, comprising the encapsulation of the ingredient in microcapsules by a method as hereinabove described, the incorporation of the microcapsules in a composition and the causing of the release of the ingredient into the environment at an appropriate time.

The granulated compositions prepared according to this invention are produced in the form of granulates having particle sizes ranging from 0.1 to 10, preferably from 0.2 to 3 and more preferably from 0.5 to 1 millimetres, and characterized by a low level, i.e. less than 5 wt %, of free-flowing fine particles and variable levels of encapsulated ingredients, i.e. from 10 wt % to 90 wt %, depending on the microcapsules to co-ingredient concentration ratio.

The composition according to the present invention is obtained by following the steps of:

1. Manufacturing a slurry, suspension or dispersion of microcapsules in water according to any process known to the art, such as coacervation, complex coacervation, interfacial polymerization (especially polycondensation), emulsion polymerization, or polymer phase separation, optionally followed by cross-linking;
2. Adding co-ingredients, such as disintegrating agents, solubilizing agents, surface-active agents, deposition promoters or film-forming agents, as hereinabove described, into said suspensions or dispersions;
3. Spraying said suspensions on to a powder bed as hereinabove described. This step leads to the spontaneous formation of “pre-agglomerates”, as hereinabove described;
4. Conveying the powder bed containing the pre-agglomerates into a drying oven heated at a temperature ranging from 50 to 200° C. to form dry agglomerates; and
5. Separating the dry agglomerates from the drying bed by sieving.

It is undesirable to have more than a small proportion of fine particles in compositions. Granulated materials obtained by performing steps 1 to 5 do not contain more than 5 wt % free-flowing fine particles and can have structures of surprisingly different sizes, shapes and appearances, depending on the nature of the powdered material used as drying bed.

In a preferred embodiment of the invention, the pre-agglomerates have particle sizes smaller than 1.5 mm, preferably smaller than 1 mm, and exhibit an excellent ability to mix homogeneously in consumer products available in granulated form, such as detergent powders, tablet bases and solid softeners.

Powder bed materials especially suitable for the formation of small, sub-millimetric and spherical pre-agglomerates from aqueous slurries or dispersions are materials having a low to moderate powder surface tension combined with zero to slow water uptake kinetics. The water uptake kinetics can be estimated by measuring the time dependence of the contact angle. For the purposes of this invention, a powder surface has the desired slow water uptake kinetics if there is exhibited an initial contact angle of at least 40°, preferably at least 50° and more preferably at least 60°, which contact angle does not change by more than 10% within the first 3 seconds of application of slurry to powder material.

The contact angle is a measure of the surface tension of the material, and it is obtained on initial contact (see D. Fennell Evans & H. Wennerstoem. “The Colloidal Domain”. VCH Pub. Inc. 1994, p. 43 ff).

Preferred drying bed materials for the manufacturing of sub-millimetric and spherical agglomerates include guar flour, gum Arabic (e.g. gum Arabic Seyal™ E 414, ex Kerry Ingredients), hydroxypropylcellulose (e.g. ex Hercules), hydroxypropylmethyl cellulose (e.g. Taian Ruitai Cellulose Co. Ltd), ethylcellulose (e.g. EC-N/NF, ex Hercules, or NF, ex The Dow Chemical Company), sodium caseinate, or carboxymethylcellulose (e.g. ex Univar). Powders of essentially hydrophobic synthetic polymers, such as polyethylene, polyamide, polyester and silicone resins are also suitable. Hydroxypropylmethyl cellulose is especially preferred.

In another preferred embodiment of the invention, there is provided water-dispersible frangible microcapsules that are electrically charged when dispersed in water and have an absolute zeta potential. In a further preferred embodiment, the invention provides microcapsule agglomerates, the microcapsules having, when dispersed in deionised water, an absolute zeta-potential of from 1-100 mV, preferably 20-40 mV, and an absolute surface charge density of from 0.1-5 Coulomb/g, preferably 0.5-1.5 Coulomb/g microcapsules.

By “zeta-potential” (ζ) is meant the apparent electrostatic potential generated by any electrically charged objects in solution, as measured by specific measurement techniques. A detailed discussion of the theoretical basis and practical relevance of the zeta-potential can be found, e.g., in “Zeta Potential in Colloid Sciences” (Robert. J. Hunter; Academic Press, London 1981, 1988). The zeta-potential of an object is measured at some distance from the surface of the object and is generally not equal to and lower than the electrostatic potential at the surface itself. Nevertheless, its value provides a suitable measure of the capability of the object to establish electrostatic interactions with other objects present in the solution, such as surfactants, polyelectrolytes and surfaces.

The zeta-potential is a relative measurement and its value depends on the way it is measured. In the present case, the zeta-potential of the microcapsules is measured by the so-called phase analysis light scattering method, using a ZetaPALS instrument (ex Brookhaven Instruments Corporation). The zeta-potential of a given object may also depend on the quantity of ions present in the solution. The values of the zeta-potential specified in the present application are measured either in deionised water, where only the counter-ions of the charged microcapsules are present, or in wash liquor, where other charged species are present.

By “absolute zeta-potential” (ℑζℑ) is meant the absolute value of the zeta-potential without reference to its (positive or negative) sign. Hence, negatively-charged objects having a zeta-potential of −10 mV and positively charged species having a zeta-potential of +10 mV have the same absolute zeta-potential.

By “absolute charge density” (ℑqℑ) is meant the number of electrical charge units per gram of dry microcapsules, expressed in Coulomb/g, without the (positive or negative) sign. In the present case, the charge density is measured by titration, using a Particle Charge Detector PCD 03 pH instrument (ex BTG Mütek GmbH).

It has been found that, according to this particular preferred embodiment, the composition specifically designed for use in detergent products is capable of releasing electrically-charged microcapsules characterized by a positive zeta-potential in deionised water and by a negative zeta-potential in wash liquors. This charge inversion is a surprising and unexpected feature of this invention. More surprising is the fact that, despite this charge inversion, i.e. despite the disappearance of the cationic character of the microcapsules in wash liquor, these microcapsules deposit very well on to fabrics during the wash cycle and are retained throughout the rinse and drying cycles, something that, in theory, should not happen. The superior deposition is inferred from the pronounced release, after drying, of microencapsulated ingredient on gentle friction, for example, by folding or unfolding fabrics, drying with towels, putting on or taking off clothes or even simply wearing clothes and moving around in them. This findings suggest that other mechanisms of deposition are involved in the present case, which were not anticipated in the prior art.

The composition according to this embodiment of the invention is characterized by its ability to deliver microcapsules having an absolute zeta-potential higher than 20 mV and lower than 40 mV and an absolute surface charge density higher than 0.5 C/g and lower than 1.5 C/g when dispersed in deionised water, whereas the sign of the electrical charge, as measured in deionised water, is selected such that it is opposite to the charge of the main ionic surfactants present in the consumer product in which the composition is incorporated. Thus, for detergents, this electrical charge is usually positive (as detergents are generally anionic), whereas, for conditioners (generally cationic), this electrical charge is generally negative.

These microcapsules are further characterized by their ability to undergo an electrical charge inversion when dispersed in wash and rinse liquors, where they acquire an electrical charge of the same sign as that of the main ionic surfactant present in the liquor. Hence, in detergent liquors containing essentially anionic and non-ionic surfactants, the microcapsules acquire a negative zeta potential, and, in conditioning liquors containing essentially cationic actives, the microcapsules acquire a positive zeta potential.

The composition according to this preferred embodiment is obtained by following the steps of:

1. Manufacturing slurries, suspensions or dispersions of microcapsules in water according to any process known to the art such as coacervation, complex coacervation, interfacial polymerization (especially polycondensation), emulsion polymerization, and more generally any kind of process known as polymer phase separation, optionally followed by cross-linking;
2. Converting said suspensions of microcapsules into suspensions of electrically charged microcapsules by adsorbing polyelectrolytes on to the surface of these microcapsules. After conversion, the microcapsules have preferably an absolute zeta potential ranging from +20 mV to +40 mV and an absolute surface charge density ranging from +0.5 C/g to +1.5 C/g after dilution in deionised water;
3. Adding co-ingredients, such as disintegrating agents, solubilizing agents, surface-active agents, deposition promoters or film-forming agents, selected from previously-mentioned list, into said suspensions or dispersions;
4. Spraying said suspensions on to a powder bed as hereinabove described. This step leads to the spontaneous formation of partially-dried pre-agglomerates, as hereinabove described;
5. Conveying the powder bed containing the pre-agglomerates into a drying oven heated at a temperature ranging from 50 to 200° C. to form dry agglomerates; and
6. Separating the dry agglomerates from the powder bed by sieving.

Polyelectrolytes suitable for converting the microcapsules into electrically-charged microcapsules (step 2) include polyanions, such as polycarboxylic acids, partially hydrolysed copolymers of (meth)acrylic acid and maleic acid or polyvinyl sulfonates, and polycations, such as cationic celluloses, guar gums or chitosan, or polymers containing quaternized amine or imidazoline groups. Polymers having the property of displaying a pH-dependent electrical charge, such as proteins and polyalkylene imines, can also be used.

Granulated materials obtained by performing steps 1 to 6 do not contain more than 5 wt % free-flowing fine particles and can have a variety of structures having surprisingly different sizes, shapes and appearances, depending on the nature of the material of the powder bed.

When added to deionised water, wash and rinse liquors, the compositions of the present embodiment, per se or as admixture with detergent products, are easily dispersed and release electrically-charged microcapsules.

In a preferred embodiment of the present invention, microcapsules are manufactured according to FR 2 663 863. The resulting slurry has a solid content ranging from 30 wt % to 40 wt %.

Alternative microcapsule manufacturing processes are also possible, for example, those disclosed in EP 026914 or EP 0978312.

In another preferred embodiment of the present invention, microcapsules are converted into positively-charged microcapsules, for example, according to the method described in FR 2 801 811. During this particular process, a positively-charged polyelectrolyte is adsorbed on to the microcapsules, which are thereby converted into positively-charged microcapsules having typically a zeta potential equal to +30+/−10 mV, as measured after dilution in deionised water.

The resulting slurry of positively-charged microcapsules is sprayed on a bed of hydroxypropylcellulose by any convenient means, typically by means of a pipette, a nozzle, a two-fluid nozzle, or a vibrating nozzle, to produce an agglomerated material having a particle size ranging from 0.1 to 10 mm, preferably from 0.2 to 3 mm and more preferably from 0.2 to 1 mm, and having a perfume loading of from 10 wt % to 90 wt %.

In another preferred embodiment, 1-30 wt % aceto-acetylated reactive polyvinyl alcohol, (available, for example, under the trade name Gohsefimer™ Z 100 or Gohsefimer Z 200 (ex Nippon Gohsei)), is used. In particular, it has been found that adding Gohsefimer Z 200 to the slurry surprisingly decreases drastically the amount of formaldehyde released over time from the aminoplast microcapsules after the slurry has been spray-dried. The invention therefore also provides a granulated composition comprising from 10 to 98 wt % water-dispersible and frangible aminoplast microcapsules that contain an ingredient for release into an environment; the composition additionally comprising from 1-30% by weight of an aceto-acetylated polyvinyl alcohol.

In another preferred embodiment of the invention, the co-ingredient is a water-soluble or water-dispersible filler material used in order to reduce the level of microcapsules in the granulated composition. Decreasing the level of microcapsules simultaneously improves (i) the dispersion of the microcapsules in the wash or rinse liquor and (ii) favours homogeneous distribution of the encapsulated active ingredients within the product. Typically, 10 to 80 wt % of such filler material is used, leading to levels of active ingredients ranging from 10 to 70 wt %.

If other components such as softeners and fabric and hair care conditioners are required, the procedure for their addition is essentially the same as that described in the embodiment specific to detergents but, in this case, in step 2 in the manufacturing process, a negatively-charged polyelectrolyte is used.

In a further preferred embodiment of the present invention, the micro-encapsulated ingredient is a fragrance material or a mixture of fragrance materials. Fragrance materials for use in compositions of the present invention may be selected from natural products such as essential oils, absolutes, resinoids, resins, concretes, and synthetic perfume components such as hydrocarbons, alcohols, aldehydes, ketones, ethers, acids, acetals, ketals and nitrites, including saturated and unsaturated compounds, aliphatic, carbocyclic and heterocyclic compounds, or precursors of any of the above. Other examples of odorant compositions which may be used are described in H 1468 (United States Statutory Invention Registration).

The amount of fragrance that can be encapsulated into the microcapsules may be up to 90 wt %, e.g. 1 to 90 w % based on dry material, with a micro-encapsulation yield close or superior to 80 wt %, even for the very volatile components having a Loss Factor of greater than 10² Pa ppm.

The term “Loss Factor” refers to a parameter that is related to the losses of fragrance material during drying and is defined as the product of the pure component vapour pressure (Pa) and the water solubility (ppm) at room temperature. Vapour pressures and water solubility data for commercially-available fragrance components are well known and so the Loss Factor for a given fragrance component may be easily calculated. Alternatively, vapour pressure and water solubility measurements may be easily taken using techniques well known in the art. Vapour pressure of fragrance components may be measured using any of the known quantitative headspace analysis techniques, see for example Mueller and Lamparsky in Perfumes: Art, Science and Technology, Chapter 6 “The Measurement of Odors” at pages 176-179 (Elsevier 1991).

The water solubility of fragrances may be measured according to techniques known in the art for the measurement of sparingly water-soluble materials. A preferred technique involves the formation of a saturated solution of a fragrance component in water. A tube with a dialysed membrane is placed in the solution such that after equilibration an idealised solution is formed within the tube. The tube may be removed and the water solution therein extracted with a suitable organic solvent to remove the fragrance component. Finally the extracted fragrance component may be concentrated and measured, for example using gas chromatography. Other methods of measuring fragrances are disclosed in Gygax et al, Chimia 55 (2001) 401-405.

The fragrance can be admixed with a texturing material or a solubilizing material prior to microencapsulation. The function of the texturing material is to increase the viscosity of the fragrance and to stabilize the microcapsules during its formation. Typical texturing materials include for example hydrophobized silicates, natural and synthetic rubber (e.g. poly(styrene-block-butadiene) and poly(styrene-block-isoprene) or polypropylene glycol. The function of the solubilizing material is to improve the affinity of the fragrance for the inner phase of the microcapsule. Typical solubilizing materials include vegetable oils such as mygliol, and silicone oils.

The amount of fragrance composition employed in perfumed products or articles according to the present invention may vary according to the particular application in which it is employed and on the fragrance loading in the fragrance composition. For detergent applications, fragrance compositions may be employed in amounts from 0.01 to 3% by weight of fragrance material based on the total weight of the detergent.

The compositions according to the invention are especially useful in personal care and household, washing and cleaning products, such as laundry detergents, solid fabric conditioners, pet litters, carpet cleaners and the like. The invention therefore provides a personal care product, a household product, a washing product or a cleaning product, comprising a composition that comprises microcapsules as hereinabove defined.

The invention is now further described with reference to the following non-limiting examples, which describe preferred embodiments.

### EXAMPLE 1

### Preparation of Electrically Charged Aminoplast Microcapsules Containing a Fragrance

Microcapsules are prepared according to the procedure described in EP 0978312 with the hydrophobic active ingredient being a test fragrance composition as described in Table 1. The microcapsules are cationized according to the procedure described in FR 2 801 811.

TABLE I

| Ingredient Name | Part |
|---|---|
| Benzyl acetate | 10 |
| Citronellyl acetate | 7.5 |
| Hexyl acetate | 5 |
| Para tert butyl cyclohexyl acetate | 4 |
| Terpenyl acetate | 15 |
| Verdyle acetate | 30 |
| Cinnamyl aldehyde | 2.2 |
| C11 undecylenyl aldehyde | 1.1 |
| C12 lauryl aldehyde | 1.5 |
| Hexyl cinnamyl aldehyde | 13.8 |
| Benzyl acetone | 7.5 |
| Citronellol | 8 |
| Cyclal C | 4 |
| Diphenyl oxyde | 10 |
| Eugenol | 5 |
| Beta Ionone | 10 |
| Alpha irisone | 25 |
| Lilial | 20 |
| Linalol | 5 |
| Ethyl methyl-2-butyrate | 3 |
| Amyl salicylate | 5 |
| Hexyle salicylate | 8.5 |
| Tetrahydro linalol | 20 |
| Undecavertol | 2.5 |
| Yara Yara | 10 |
| | 237.1 |

The slurry obtained in example 1 has a solid content of 35±5 wt % and a perfume content of 27±3 wt %.

### EXAMPLE 2

### Preparation of Granules Containing Electrically-Charged Microcapsules

10 parts of citric acid is added to the 90 parts of slurry obtained in Example 1 and the resulting mixture is sprayed on a bed of hydroxypropyl methyl cellulose (HPMC) by using a pipette with an inner diameter of 1 mm. The obtained granules are then directly dried in an oven at 50-70° C. for 30 minutes and once dried the HPMC is removed by sieving.

The resulting granules have particle sizes of 3±1 millimeters, are water soluble and have a total oil content ranging from 85±5 wt %, as measured by pulsed NMR method using an Oxford MQA6005 (Oxford Instruments IAG, UK), reflecting that close to 100 wt % of the perfume is present in liquid form in the microcapsules.

### EXAMPLE 3

### Wash Test

The composition according to Example 1 is mixed in standard, non-perfumed detergent powder base at 0.3 wt % and 1% perfume level, and 100% perfume encapsulated. Machine wash conditions: 220 g towels, 32 g wash powder, ca. 10 L water, wash temperature 40° C.

Olfactory evaluation is performed by 6 trained panelists. Assessment is made after drying (line-dried towel, before and after gentle rubbing). Intensity scale: 0=imperceptible, 1=very weak, 2=weak, 3=medium, 4=strong, 5=very strong.

The impact of the composition is clearly stronger than that of the corresponding free oil

Olfactory evaluation on wet and dry towels after 5 days

Panel: 6 people.

| | granulated | free |
|---|---|---|
| | fragrance | fragrance |
| with rubbing | 2.2 | 0.2 |
| without rubbing | 0.3 | 0.25 |

### EXAMPLE 4

### Measurement of Contact Angle

Powder-water contact angle measurements of various powder bed materials were performed using a contact angle meter CAM-100 (KSV Ltd). For this, each powder bed material was tabletized and one droplet of water was dropped onto the tablets using a Hamilton pipette. 10 pictures were taken for every droplet within 1 second, from which the mean value of the contact angle was obtained (Table 2). The procedure was repeated 5 times within 5 seconds to yield the time-dependence of the contact angle during the first 3 seconds.

Materials having a time-dependence of the contact angle smaller than 10% within the first 3 seconds are designated as “slow”, the others as “fast”.

TABLE 2
Contact angle measurement of one droplet of water on different powders.

| | Measured contact | Water uptake | |
|---|---|---|---|
| | angle¹ | kinetics | Observation |
| Silicone resin | >70° | Slow | Stable droplet, constant |
| | | | contact angle |
| Ethyl cellulose | 77.6° | Slow | Stable droplet, contact |
| | | | angle decreases slightly |
| | | | with time |
| Hydroxypropyl methyl | 63.4° | Slow | Stable droplet |
| cellulose (HPMC) | | | |
| Guar flour | 48.8° | Limit | Quite stable droplet but |
| | | | the sample swells |
| Microcrystalline | 30.8° | Slow | Quite stable droplet but |
| Cellulose | | | the sample swells |
| Starch 1415 | 25.1° | Fast | Droplet flattening, |
| | | | contact angle decreases |
| | | | rapidly with time |
| Kieselgel 60 | Not measurable | Fast | Water is absorbed by the |
| | | | sample |

¹Mean value of 10 measurements.

### EXAMPLE 5

### Manufacturing of Agglomerates with Various Shape Morphology and Dimensions

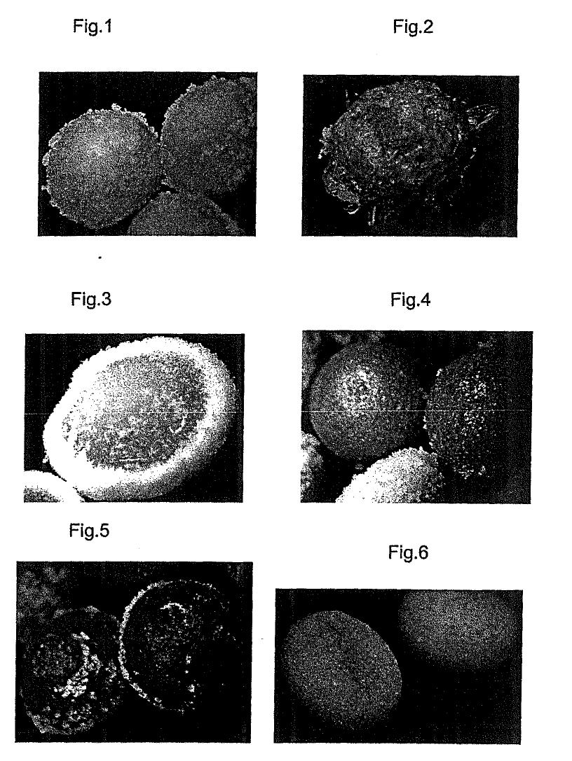

Slurries prepared accordingly with Example 1 are deposited on to various powder beds characterized by various contact angles and water uptake kinetics, using a pipette with inner diameter 1 mm. The resulting shapes and morphologies are depicted in FIGS. 1-6

The details of the figures are as follows:

| Figure | | |
|---|---|---|
| No. | powder | description |
| 1 | gum Arabic | semi-round, (size: 2-5 mm) |
| 2 | hydroxypropylcellulose | round, slightly warped surface |
| | | (size: 2-5 mm) |
| 3 | dexylose | “fried egg”-like (size: 2-5 mm) |
| 4 | sodium caseinate | semi-circular (size: 2-5 mm) |
| 5 | hydroxypropyl methyl | round and compact (size 2-3 mm) |
| | cellulose | |
| 6 | ethyl cellulose | flat (size 2-3 mm) |

The contact angle is measured on a drop deposed onto the powder bed, using an optical contact angle measuring system CAM 100 (ex KSV Ltd). The decay of the contact angle as a function of time (in degrees per second) is taken as assessment of the water uptake kinetics.

### EXAMPLE 6

### Manufacturing of Agglomerates Having Sizes Smaller than 1 mm

Slurries prepared accordingly to Example 1 are sprayed on to a rotating HPMC bed operating at 90 rpm, by using a two fluid vibrating nozzles operating at 400 Hz and having an inner diameter of 0.6 mm. The obtained pre-agglomerates are then dried on the HPMC bed in an oven at 60° C. for 30 minutes and then sieved. The resulting granules have spherical to short worm-like shape and a mean diameter smaller than 1 mm.

1. A method of making a granulated composition that comprises frangible microcapsules containing at least one active ingredient, comprising the step of:
spraying of an aqueous slurry of the microcapsules on to a non-fluidised bed of powdered material and then drying, the powdered material being hygroscopic and having a powder surface tension as defined by an initial contact angle of slurry with powder of at least 40°, which contact angle does not change by more than 10% within the first 3 seconds of application of slurry to powder material.

2. A method according to claim 1 in which the initial contact angle is at least 50°.

3. A method according to claim 1, in which the microcapsules are electrically charged, prior to their application to the bed of powdered material, by having polyelectrolytes adsorbed on to the surface of the microcapsules.

4. A method according to claim 3, in which the microcapsules have an absolute zeta potential of from +20 mV to +40 mV and an absolute surface charge density of from +0.5 C/g to +1.5 C/g after dilution in deionised water.

5. A process of releasing into an environment in a controlled manner an ingredient whose presence in that environment is desired, comprising the steps of: encapsulation of the ingredient in microcapsules, incorporation of the microcapsules in a composition and causing the release of the ingredient into the environment at an appropriate time, characterised in that
the microcapsules have, when dispersed in deionised water, an absolute zeta-potential of 1-100 mV, and an absolute surface charge density of from 0.1-5, Coulomb/g microcapsules.

6. A granulated composition of frangible microcapsules, containing at least one active ingredient formed by a process which includes the step of:
spraying of an aqueous slurry of the microcapsules on to a non-fluidised bed of powdered material and then drying, the powdered material being hygroscopic and having a powder surface tension as defined by an initial contact angle of slurry with powder of at least 40°, which contact angle does not change by more than 10% within the first 3 seconds of application of slurry to powder material.

7. A granulated composition according to claim 6, in which the microcapsules additionally comprise from 2-90 wt % of at least one co-ingredient selected from water-soluble filler material, surfactant, disintegrating agent, solubilising agent, hydrocolloid, film forming agent and a deposition promoter.

8. A granulated composition according to claim 6, in which there is present from 2-80 wt % of at least one co-ingredient.

9. A granulated composition according to claim 6, in which the microcapsules have, when dispersed in deionised water, an absolute zeta-potential of from 1-100, mV and an absolute surface charge density of from 0.1-5, Coulomb/g microcapsules.

10. A granulated composition comprising from 10 to 98 wt % water-dispersible and frangible aminoplast microcapsules that contain an ingredient for release into an environment; the composition additionally comprising from 1-30% by weight of an aceto-acetylated polyvinyl alcohol.

11. A granulated composition according to claim 10, in which the microcapsules are obtained by interfacial copolymerization of melamine or melamine pre-polymer with formaldehyde or glutaraldehyde.

12. A product selected from the group which comprises: a personal care product, a household product, a washing product or a cleaning product, wherein said produce comprises a granulated composition according to claim 6.

14. A product selected from the group which comprises: a personal care product, a household product, a washing product or a cleaning product, wherein said produce comprises a granulated composition according to claim 7.

15. A product selected from the group which comprises: a personal care product, a household product, a washing product or a cleaning product, wherein said produce comprises a granulated composition according to claim 8.

16. A product selected from the group which comprises: a personal care product, a household product, a washing product or a cleaning product, wherein said produce comprises a granulated composition according to claim 9.

17. A product selected from the group which comprises: a personal care product, a household product, a washing product or a cleaning product, wherein said produce comprises a granulated composition according to claim 10.

18. A product selected from the group which comprises: a personal care product, a household product, a washing product or a cleaning product, wherein said produce comprises a granulated composition according to claim 11.

19. A process of releasing into an environment in a controlled manner an ingredient whose presence in that environment is desired according to claim 5 comprising the steps of: encapsulation of the ingredient in microcapsules, incorporation of the microcapsules in a composition and causing the release of the ingredient into the environment at an appropriate time, characterised in that:
the microcapsules have, when dispersed in deionised water, an absolute zeta-potential of from 20-40 mV and an absolute surface charge density of from 0.5-1.5 Coulomb/g microcapsules.

20. A granulated composition according to claim 9, in which the microcapsules have, when dispersed in deionised water, an absolute zeta-potential of from 20-40, mV and an absolute surface charge density of from 0.5-1.5, Coulomb/g microcapsules.
